(19) United States
(12) Patent Application Publication
(10) Pub. No.: US 2026/0295216 A1
(43) Pub. Date: Oct. 1, 2026

(54) CATHETERIZATION APPARATUS AND METHOD OF USE

(71) Applicant: TELEFLEX MEDICAL LLC, Morrisville, NC (US)

(72) Inventors: Benjamin Horst, Lititz, PA (US); Jeffrey Kuehn, Schuylkill Haven, PA (US); Jason Beissel, Bernville, PA (US)

(21) Appl. No.: 19/631,665

(22) Filed: Mar. 27, 2026

Related U.S. Application Data
(60) Provisional application No. 63/779,990, filed on Mar. 28, 2025.

(51) Int. Cl. A61M 25/01; A61M 25/00; A61M 25/06

(52) U.S. Cl. CPC A61M 25/0113; A61M 25/0097; A61M 25/0102; A61M 25/0606; A61M 25/0693; A61M 2205/0216; A61M 2205/583; A61M 2210/12

(57) ABSTRACT

A catheter insertion device and a method of operating the device is disclosed. The device includes an inner body, a needle attached to the inner body, and an outer body. Moreover, the device includes a guidewire fixed to a proximal end of the outer body and a stiffening member fixed to the proximal end of the outer body configured to stabilize the guidewire when the outer body moves between the first position and the second position. Further, when the outer body moves between the first position and the second position, the outer body slides over the inner body and the guidewire and the stiffening member simultaneously move through an inner portion of the inner body. Additionally, when the outer body is at the first position, a tip of the guidewire is within the needle and at the second position, this tip is advanced into a vessel of a patient.

### CROSS-REFERENCE TO RELATED APPLICATIONS

This application claims benefit of priority to U.S. Provisional Application No. 63/779,990, filed on Mar. 28, 2025, the entire contents of which are incorporated by reference herein.

### FIELD OF THE INVENTION

The present invention generally relates to a catheterization apparatus capable of providing continuous blood flashback during insertion of a catheter into vasculature of a patient, and more particularly, to an over-the-needle catheter insertion device having an integrated guidewire with and without a needle safety clip.

### BACKGROUND

Catheters are commonly used to remove fluids from blood vessels of a patient, or introduce fluids into blood vessels, for a variety of medical procedures. For instance, radial artery catheterization may be performed to provide access to the arterial system via the radial artery for monitoring blood pressure and withdrawing blood. Catheters are often inserted into the vasculature via a catheter insertion device. In a typical catheterization procedure, for instance, in order to insert a catheter in a vessel, the vessel access is first performed by using a long hollow needle to pierce the vessel wall. A guidewire is then passed through the needle and into the vessel. The guidewire acts as a track for the catheter to pass over to reach a target location within the vessel. A catheter is finally passed over the guidewire to the target location in the vasculature of the patient. With the catheter in place, the needle and the guidewire are removed, leaving only the catheter within the vessel. Fluids may then be introduced into, or removed from, the vessel through the catheter by connecting a fluid source or aspiration device to the catheter hub or the catheterization device may be connected to blood pressure monitoring equipment.

Conventional catheterization devices include a means for the practitioner to visualize blood flash (i.e., the return of blood in the body of the device before guidewire advancement) for indicating when the tip of the needle has entered the vasculature during vessel puncture. Some known catheterization devices offer the ability for users to visualize pulsatile blood flash when the vessel, such as an artery, is initially punctured. These devices allow the practitioner to confirm vessel puncture during insertion and how centered the needle is within the vessel, or if the needle was inadvertently removed from the vessel after the initial puncture, by judging the rate of blood flash. However, many devices do not permit pulsatile blood flash that remains visible after guidewire advancement.

Rather, the blood flash in conventional catheter insertion devices stops during and after the subsequent guidewire advancement step because the guidewire occupies too much of the volume within the needle, thus reducing flow. For example, conventional catheter insertion devices with blood flash have a very small space or annular cross-sectional area between an outer diameter of the guidewire and the inner diameter of the needle, which results in not enough blood flow to allow users to actually see the flash if the wire is advanced into the needle. Further, guidewire advancers in conventional catheter insertion devices are often placed far from the grip position on the device (i.e., have a large guidewire reach length) and start near the back of the needle (i.e., have a large guidewire advancement dead length) and thus take longer for the guidewire to enter the artery. This aspect makes it difficult to ensure the needle tip remains in artery for the catheter insertion device, because the user does not have a reliable way of confirming placement of the needle within the artery.

Other conventional catheterization devices offer the ability for users to visualize blood flash during vessel puncture through a transparent, or partially translucent, catheter chamber as the blood enters the small volume of space between the inside of the hollow catheter and the outside of the hollow needle. These devices show flash through the catheter when it initially enters into this small volume of space, but these devices do not offer the ability for flash to re-start after it is first observed and very quickly fills this small volume of space. For example, devices of this kind of funnel like shaped chambers which fill quickly on the distal end but then slows as the chamber fills toward the proximal end to the point where the flow rate of the blood flash is not helpful to the clinician. This aspect makes it difficult for the practitioner to recover an insertion in which the needle tip becomes misaligned with the vessel or passes through the vessel.

Even for a catheter insertion device that includes pulsatile blood flash that remains visible in the device (e.g., the guidewire tube, handle or the like) after guidewire advancement, these devices do not provide adequate continuous flash visibility for clinical use. So, even if the annular cross-sectional area was increased from the use of a smaller guidewire, and the chamber was cylindrical, rather than funnel shaped, the guidewire tube would have to be increased in length in order to allow the user to adequately see the blood flash. However, increasing the length of the device would position the location where the clinician manipulates the guidewire much further back from the point where the clinician holds the device at the catheter hub and thus increase the guidewire reach length. Thus, this increase in the guidewire reach length would make the catheter insertion device very difficult to use. Furthermore, the mechanism for wire advancement would need to ensure it didn’t allow blood to escape from the guidewire tube (e.g., if the guidewire tube had an opening or slit or the like as is on conventional devices); otherwise, it would prevent the continuous flash visibility after guidewire advancement.

Therefore, a clear need exists for a novel catheter insertion device that allows for continuous visible blood flashback during insertion of a catheter into the vasculature of a patient. More particularly, there exists a need for a catheterization device that offers the ability to visualize flash through the catheter and flash in the device (i.e., the guidewire tube or the like) (which also includes the ability to re-confirm flash in guidewire tube or the like), and also offer the ability to visualize flash during and after guidewire advancement.

There is also a need for a catheterization device that offers the ability to visualize blood flash in the guidewire tube or the like (which also includes the ability to re-confirm flash in the guidewire tube or the like) and also start the guidewire inside the needle and near the needle tip, which both reduces guidewire reach length and reduces guidewire advancement dead length. These features thus improve the ease and speed of catheterization by allowing the guidewire to enter and secure the artery for catheterization and offer visual feedback to the user through blood flash. Additionally, a need exists for a catheter insertion device that allows for intuitive, easy, safe, and fast catheter placement into a patient's vasculature.

Moreover, known guidewire advancers may require a significant force to actuate the advancer to move the guidewire forward, which may cause the device to be dislodged or without a tactile feel to aid the clinician in accurately placing the guidewire in the vessel. Other guidewire advancers may rather allow the guidewire to freely slide, so if the clinician holds the device upright before the needle punctures the skin, the guide wire may fall forward from gravity which may increase the likelihood of damage to the patient’s skin or the catheter insertion device. Thus, a need exists for a catheter insertion device that provides for stable and reliable catheter placement into a patient’s vasculature by providing a sufficient amount of resistance to the guidewire during guidewire advancement to ensure the guidewire is not free to slide but also provides enough of a tactile feel to the clinician to prevent dislodging.

Additionally, in clinical practice, injuries from needles are dangerous because of blood and infective agents that contaminate the used needle. These injuries, which are occupational hazards of the medical profession, have resulted in inadvertent transmission of infective agents such as hepatitis C and human immunodeficiency virus. Sharps collection boxes, gloves and various safety practices are used to mitigate injuries arising from used needles, and to prevent infections from the needles. However, the presently available safety devices for protecting against injuries and infections resulting from needles are not completely fail-safe. Moreover, radial artery catheters often have integrated guidewires which many existing needle safeties do not work with. Furthermore, conventional safety clips that work with guidewires are often too large and thus cause the needle housing to be too big or bulky, which provides for poor device ergonomics and increases the likelihood of adverse behavior of the safety clip during catheter insertion. Therefore, the present disclosure addresses these unmet needs by providing a new safety clip for a needle that is compatible for use with a guidewire, provides for improved ergonomics, minimizes the risk of poor safety clip performance, and is easy to manufacture.

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

The foregoing needs are met, to a great extent, by the present disclosure of a catheter insertion device discussed herein. For example, a catheter insertion device may include an inner body defining a continuous pulsatile blood flashback chamber. The catheter insertion device may also include a needle hub having a proximal end attached to a distal end of the inner body and a hollow introducer needle extending from a distal end of the needle hub, where the needle may have a tip configured to pierce a wall of a blood vessel. Moreover, a catheter may be configured to slidably fit over the introducer needle and extend from a catheter hub. Additionally, the catheter insertion device may have an outer body configured to slidably receive a portion of the inner body, a guidewire coupled to a proximal end of the outer body, and a stiffening member extending from the proximal end of the outer body.

Moreover, the outer body may be further configured to move between a first position in which the guidewire is at an undeployed state and a second position in which the guidewire is at a deployed state. The tip of the guidewire may be located within the needle when the outer body is at the first position and the tip of the guidewire may be advanced beyond the tip of the needle when the outer body is at the second position.

The described implementations of the invention may also include one or more of the following features. For example, the guidewire and the stiffening member may simultaneously move when the outer body moves between the first position and the second position, such that the stiffening member may stabilize a portion of the guidewire. The catheter insertion device may also include a stabilizer attached to a proximal end of the inner body, which may be configured to receive the stiffening member and provide friction to the stiffening member when the outer body moves between the first position and the second position. The stabilizer may include an elastomeric disk having an elliptical or oval shaped aperture configured to receive the stiffening member and provide friction to the stiffening member when the outer body moves between the first position and the second position.

In one implementation, the stabilizer may include a rudder, the outer body may include an elongated slot, and the rudder may be configured to slide within the slot to prevent or minimize rotation of the outer body relative to the inner body when the outer body moves between the first position and the second position. Moreover, the outer body further may include a stop member at a distal end of the elongated slot configured to abut the rudder of the stabilizer to prevent the outer body from moving proximally relative to the inner body. The stop member may also include a pair of protrusions extending into the slot, the pair of protrusions forming a passage to an open distal end of the slot and the rudder of the stabilizer may be configured to pass through the passage of the slot upon application of a threshold force against the pair of protrusions to allow the outer body to detach from and/or attach to the inner body.

In another implementation, the stiffening member may be a hypotube configured to receive the portion of the guidewire. In another implementation, the stiffening member may be a proximal portion of the guidewire having a diameter that is larger than a diameter of a distal portion of the guidewire.

Moreover, a distal end of the outer body may include a guidewire actuator configured to move the guidewire between the undeployed state and the deployed state. The distal end of the guidewire actuator may also include a flag configured to allow a user to move the outer body between the first position and the second position. The proximal end of the guidewire actuator may be over-molded or otherwise attached onto a distal end of the outer body and the distal end of the stiffening member may be located below the flag to create a push point for the user. The guidewire actuator may furthermore include at least one projection configured to contact the inner body to minimize friction when the outer body is moved between the first position and the second position. In one implementation, the catheter insertion device may include a safety clip configured to provide sharps protection to the tip of the introducer needle upon removal of the catheter from the introducer needle.

The present disclosure also relates to a method of operating a device capable of continuous pulsatile blood flash to advance a guidewire fixed to a distal end of an outer body of the device into a vessel of a patient. For example, the method may include moving the outer body of the device to a first position, inserting a needle attached to a proximal end of an inner body of the device into the vessel of the patient, and moving the outer body from the first position, where a tip of the guidewire is within the needle, to a second position, where the tip of the guidewire is advanced into the vessel of the patient. Further, when the outer body moves between the first position and the second position, the outer body may slide over the inner body and the guidewire and a stiffening member fixed to the distal end of the outer body may simultaneously move through an inner portion of the inner body.

The guidewire and the stiffening member may also extend through a hole of a stabilizer fixed to a distal end of the inner body and the stiffening member may be configured to stabilize the guidewire when the outer body moves from the first position to the second position. The stiffening member may include a hypotube configured to receive the portion of the guidewire. The stiffening member may include a portion of the guidewire having a diameter that is larger than a diameter of a distal portion of the guidewire. The device may further include an elastomeric disk having an elliptical or oval shaped aperture configured to receive the stiffening member and provide friction to the stiffening member when the outer body moves between the first position and the second position. The stabilizer may include a rudder, the outer body may include an elongated slot, and the rudder may be configured to slide within the slot to prevent or minimize rotation of the outer body relative to the inner body when the outer body moves between the first position and the second position. The device further may include a safety clip configured to provide sharps protection to the tip of the introducer needle upon removal of the catheter from the introducer needle.

The safety clip may include a proximal portion comprising a first proximal wall directly connected to a second proximal wall at a clip bend, the first proximal wall defining a first proximal aperture and the second proximal wall defining a second proximal aperture, the second proximal wall being more proximal than the first proximal wall. Additionally, the safety clip may include a first distal portion including a first distal wall, the first distal wall defining a first distal aperture for receiving the needle, a first clip arm extending from the first distal portion to the first proximal wall and including a medial aperture for receiving the needle, a second distal portion comprising a second distal wall, the second distal wall defining a second distal aperture for receiving the needle.

In another implementation, the safety clip may also include a second clip arm extending from the second distal portion to the second proximal wall, the first clip arm and the second clip arm may be in contact with the needle, and the first clip arm and the second clip arm may be configured to flex between a first configuration and a second configuration to move the safety clip relative to the needle. Moreover, when the first clip arm and the second clip arm are in the first configuration, the safety clip may be able to slide along a length of the needle and the first distal wall and the second distal wall are spaced apart and when the first clip arm and the second clip arm are in the second configuration, the second proximal aperture may be configured to engage a swaged portion of the needle such that the first distal wall and the second distal wall overlap to cover a tip of the needle.

In one implementation, the first clip arm and the second clip arm may not intersect in either the first configuration or the second configuration. Further, the safety clip further may include a keeper configured to provide frictional resistance against an inner surface of the catheter hub to retain the safety clip inside the catheter hub. The catheter hub may include a bump configured to prevent the safety clip from being removed from the catheter hub in a first configuration and configured to allow the safety clip to be removed from the catheter hub in a second configuration. Further, when the safety clip reaches a bevel of a tip of the needle, the safety clip may activate and allow the first clip arm and the second clip arm to move from the first configuration to the second configuration.

### BRIEF DESCRIPTION OF THE DRAWINGS

FIG. 1 discloses a perspective view of a catheter insertion device according to the present invention in a first position.

FIG. 2 discloses a perspective view of the catheter insertion device in the second position.

FIG. 3 discloses a perspective view of the needle of the catheter insertion device being inserted into a vessel of a patient.

FIG. 4 discloses a bottom plan view of the outer body of the catheter insertion device in the first position.

FIG. 5 discloses a perspective view of the stabilizer.

FIG. 6 discloses a front view of the stabilizer.

FIG. 7 discloses a front plan view of the outer body.

FIG. 8 discloses a side plan view of the catheter hub and the needle hub.

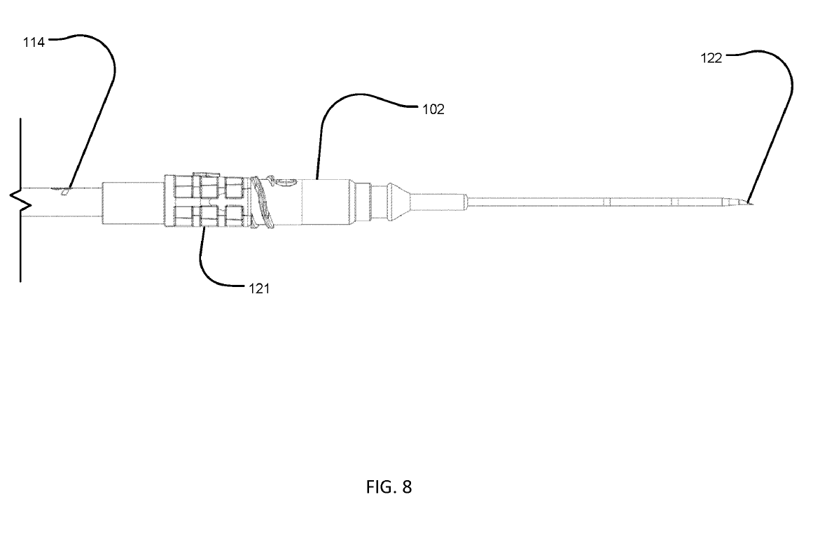

FIG. 9 discloses a side plan view of the catheter insertion device in the first position with a hypotube as the stiffening member compared to a side plan view of a thickened portion of the guidewire acting as a stiffening member.

FIG. 10 discloses an enlarged side plan view of the area indicated in FIG. 8 showing the profile of the guidewire installed in the catheter insertion device compared to a side plan view of a thickened portion of the guidewire acting as a stiffening member.

FIG. 11 discloses an enlarged side plan view of the area indicated in FIG. 8 showing the profile of the guidewire installed in the catheter insertion device compared to a side plan view of a thickened portion of the guidewire acting as a stiffening member.

FIG. 12 discloses an enlarged side plan view of the area indicated in FIG. 8 showing the profile of the guidewire and the hypotube acting as a stiffening member installed in the catheter insertion device compared to a side plan view of a thickened portion of the guidewire acting as a stiffening member.

FIG. 13 discloses an enlarged side plan view of the area indicated in FIG. 8 showing the profile of the guidewire and the hypotube acting as a stiffening member installed in the catheter insertion device compared to a side plan view of a thickened portion of the guidewire acting as a stiffening member.

FIG. 14 discloses an enlarged side plan view of the area indicated in FIG. 8 showing the profile of the guidewire and the hypotube acting as a stiffening member installed in the catheter insertion device compared to a side plan view of a thickened portion of the guidewire acting as a stiffening member.

FIG. 15 discloses a side view of the safety clip within the catheter hub of the catheter insertion device.

FIG. 16 shows a perspective view of the safety clip in the first configuration.

FIG. 17 shows a perspective view of the safety clip in the second configuration.

FIG. 18 shows a top plan view of the safety clip in the second configuration.

FIG. 19 shows a side plan view of the safety clip with a keeper in the first configuration.

FIG. 20 shows a side plan view of the safety clip with a keeper in between the first configuration and the second configuration.

FIG. 21 shows a side plan view of the safety clip with a keeper in the second configuration.

FIG. 22 shows a top plan view of the safety clip with a keeper in the second configuration.

FIG. 23 shows a perspective view of the safety clip with a keeper.

FIG. 24 shows a side plan view of the safety clip in the first, open configuration.

FIG. 25 shows a side plan view of the safety clip in the second, closed configuration.

FIG. 26 shows a perspective view of the safety clip in the first, open configuration.

FIG. 27 shows a perspective view of the safety clip in the second, closed configuration.

### DETAILED DESCRIPTION

As will be discussed in detail herein, the present disclosure describes an over-the-needle catheter insertion device operable to provide continuous pulsatile blood flashback visibility during catheterization. In particular, the catheter insertion device is operable to provide continuous pulsatile blood flashback in a guidewire passageway both during and after advancing the guidewire. The catheter insertion device is also operable to re-start pulsatile blood flashback within the guidewire passageway after initial pulsatile blood flashback within the guidewire passageway has stopped, i.e., due to misalignment of the needle tip within the vessel or due to passing the needle tip through the vessel wall. The ability to re-start pulsatile blood flashback in the guidewire passageway allows the practitioner to re-confirm proper placement of the needle tip within the patient’s vasculature. The catheter insertion apparatus is also operable to provide the continuous blood flash visibility while starting the wire guide tip inside and near the needle tip, without causing excessive blood exposure, and without impacting other critical aspects of an insertion procedure. The catheter insertion device of the present disclosure therefore improves insertion success rates and increases the confidence of practitioners in knowing that the needle tip is located inside the vessel throughout the entire insertion procedure without causing excessive blood exposure. The present catheter insertion apparatus may be utilized for catheterization of various vessels, including the radial artery or other arterial vessels, as well as the venous vasculature. Further, the present catheter insertion apparatus may be utilized to insert an introducer catheter used for facilitating the insertion of other catheters, such as a PICC (Peripherally Inserted Central Venous Catheter) or CVC (Central Venous Catheter) or other vascular access devices.

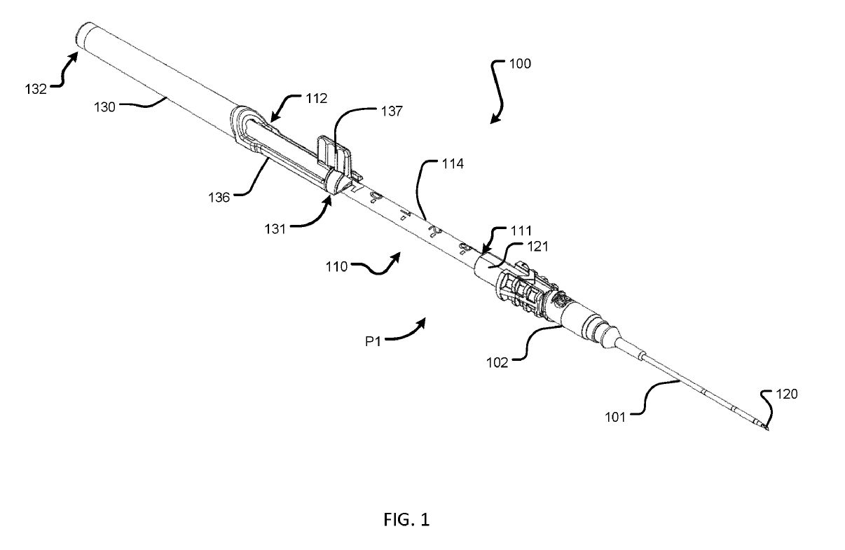

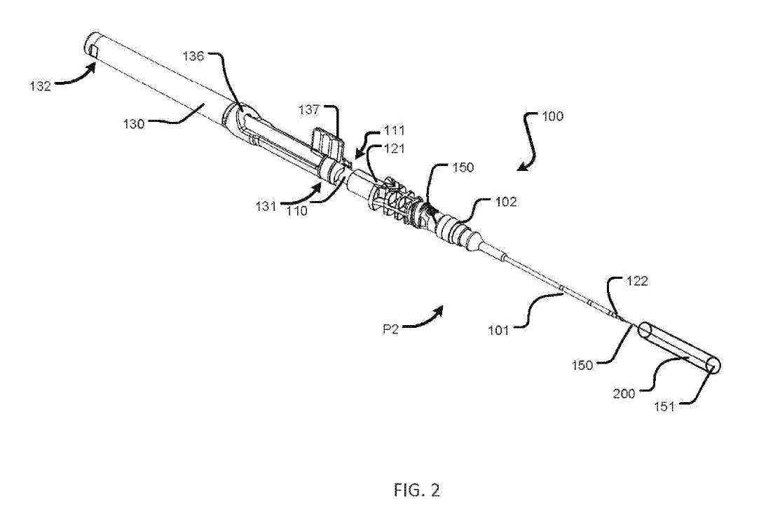

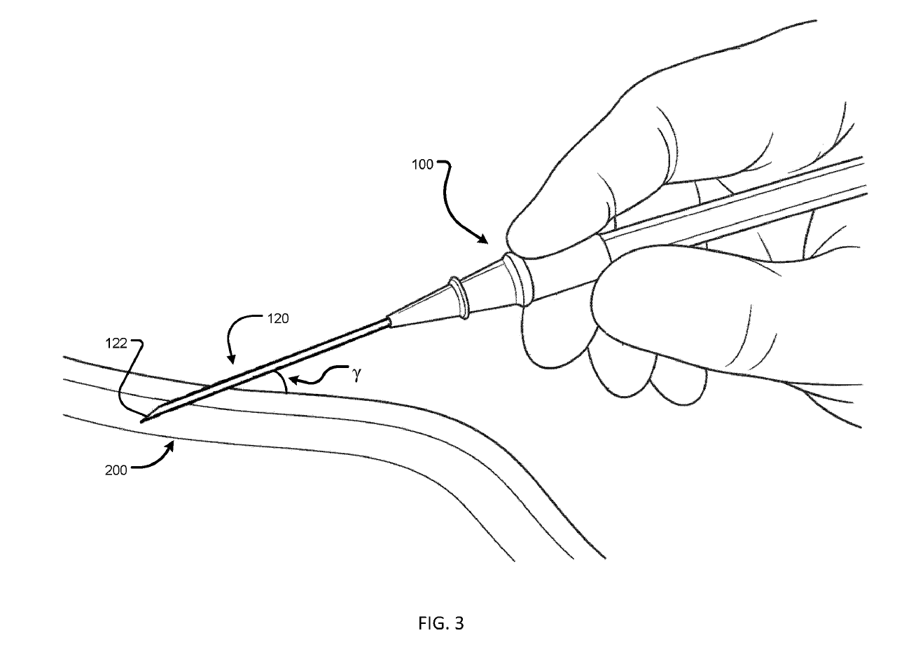

Referring to FIGS. 1 and 2, one aspect of the disclosure relates to a catheter insertion device 100 capable of continuous pulsatile blood flash which may include an inner body 110, which is designed to act as a pulsatile blood flashback chamber 114 to store blood of a patient during continuous flash. According to aspects of the disclosure, various components are identified as distal or proximal in relation to a user (e.g., a clinician or practitioner). The catheter insertion device 100 capable of continuous pulsatile blood flash may also include a hollow introducer needle 120 extending from a distal end of the needle hub 121, the needle having a tip configured to pierce a wall of a blood vessel. The catheter insertion device 100 capable of continuous pulsatile blood flash may furthermore include an outer body 130 configured to move between a first position P1 and a second position P2. In this way, the catheter insertion device 100 may be configured to provide continuous pulsatile blood flashback within the flashback chamber 114 during catheterization, including before moving the guidewire 150, while moving the guidewire 150, and after moving the guidewire 150. In some aspects, the flashback chamber 114 is transparent so that the pulsatile blood flashback is visible to the user. In some aspects, the flashback chamber may include indicia, such as spaced apart alphanumeric markings or other type of markings, to indicate a specific distance of guidewire advancement. Further, FIG. 3 illustrates an insertion location for the catheterization procedure, where a practitioner inserts the tip 122 of the needle 120 at approximately a 30° insertion angle (γ) with respect to the surface of the skin to access a vessel 200 of the patient.

Additionally, the catheter insertion device 100 may include a catheter 101 including a catheter hub 102 removably coupled to a needle hub 121 of the catheter insertion device 100 and the needle hub 121 may be fixed to the distal end of the inner body 111. For example, the catheter hub 102 may be friction fit onto the needle hub 121, while the needle hub 121 may be adhered onto the distal end of the inner body 111. Further, the catheter 101 may slidably fit over a introducer needle 120 with the catheter 101 extending from the catheter hub 102.

FIG. 1 shows the catheter insertion device 100 of the invention when it is in a first position P1. In this first position P1, the outer body 130 is at a distance extending away from the tip 122 of the needle 120 and the guidewire tip 151 is in an undeployed state located within the body of the needle 120. Additionally, in this first position P1 the distal end of the outer body 131 overlaps the proximal end of the inner body 112.

FIG. 2 shows the catheter insertion device 100 of the invention when it is in a second position P2. In this second position P2, the distal end of the outer body 131 is located close to the distal end of the inner body 111, while the proximal end of the inner body 112 is located close to the proximal end of the outer body 132. Furthermore, while the outer body 130 is in the second position P2, the guidewire 150 is in a deployed state which extends past the tip 122 of the needle 120 and into the vessel 200 of a patient.

In another aspect of the invention, the outer body 130 of the catheter insertion device 100 may be configured to slidably receive a portion of the inner body 110 between the first position P1 and the second position P2. Further, the catheter insertion device 100 capable of continuous pulsatile blood flash may include a stabilizer 140 having an aperture 141. The aperture 141 preferably may be elliptical or oval shaped, but slits, square shaped apertures, round shaped apertures, or other shaped apertures understood by those skilled in the art are contemplated. Additionally, the stabilizer 140 may be attached to a proximal end of the inner body 112. The catheter insertion device 100 may moreover include a guidewire 150 attached to a proximal end of the outer body 132, which extends through the center of the inner body 110, the aperture 141 of the stabilizer 140, and the outer body 130 and into the needle 120. The catheter insertion device 100 capable of continuous pulsatile blood flash may also include a stiffening member 160 extending from the proximal end of the outer body 132 configured to stabilize a portion of the guidewire 150 when the outer body 130 moves between the first position P1 and the second position P2.

The catheter insertion device 100 of the present disclosure may furthermore have the guidewire 150 and the stiffening member 160 extend through the aperture 141 of the stabilizer 140. Further, when the outer body 130 of the catheter insertion device 100 is at the first position P1, a guidewire tip 151 may be located within the needle 120 and when the outer body 130 is at the second position P2, the tip 151 the guidewire 150 may be advanced beyond the tip 122 of the needle.

Additionally, when the outer body 130 of the catheter insertion device 100 moves between the first position P1 and the second position P2, the outer body 130 may slidably receive a portion of the inner body 110. During this time, the guidewire 150 and the stiffening member 160 may simultaneously move through an inner portion 113 of the inner body 110 and the aperture 141 of the stabilizer 140, such that the stiffening member 160 stabilizes a portion of the guidewire.

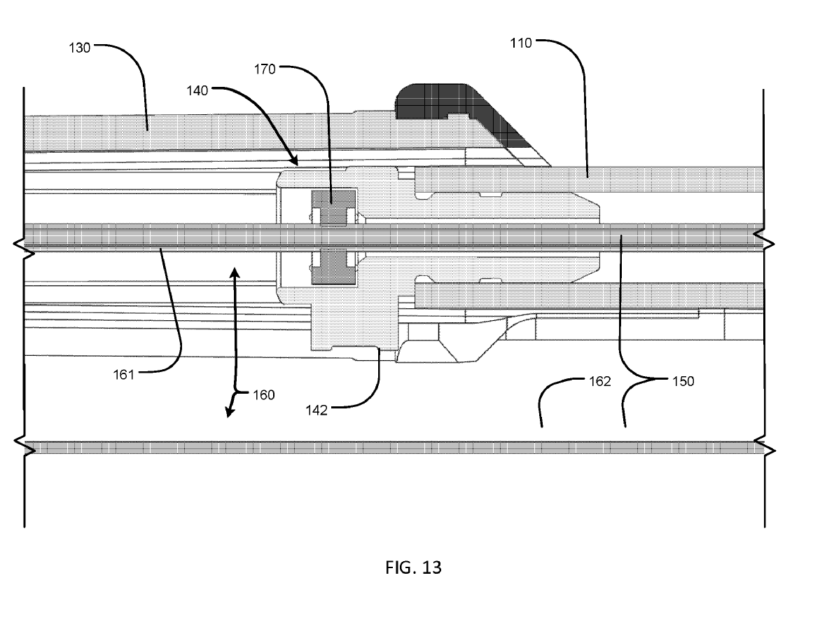

The arrangement between the outer body 130, inner body 110, stabilizer 140, guidewire 150, and stiffening member 160 allows the pulsatile blood flashback chamber 114 to substantially contain the blood of the patient inside during continuous flash. Furthermore, the needle hub 121 may be bonded to the inner body at its distal end 111, creating a fluid passageway from the tip 122 of the needle, through the needle 120, through the needle hub 121, and into the blood flashback chamber 114 of the inner body. The proximal end 112 of the flashback chamber 114 is designed with enough clearance between the stiffening member 160 and the aperture 141 of the stabilizer 140 so that air may escape, but not too much clearance so that blood can easily exit the inner body 110, such as seen in FIG. 13. In most cases, blood will not be present outside of the catheter insertion device 100, but when the inner body 110 is fully filled with blood, some blood may weep out of the inner body chamber. The elliptical or oval shape of the stabilizer’s aperture 141 also helps to create a pressure differential within the inner body 110 to allow the blood to flash back and flow into the blood flashback chamber 114 of the inner body. Preferably, the clearance between the stiffening member 160 and the aperture 141 of the stabilizer 140 is approximately 0.0015 inches or between 0.0014 inches and 0.0016 inches. Thus, the stabilizer 140 functions additionally as a plug attached to the proximal end of the inner body 112 for the blood flashback chamber 114.

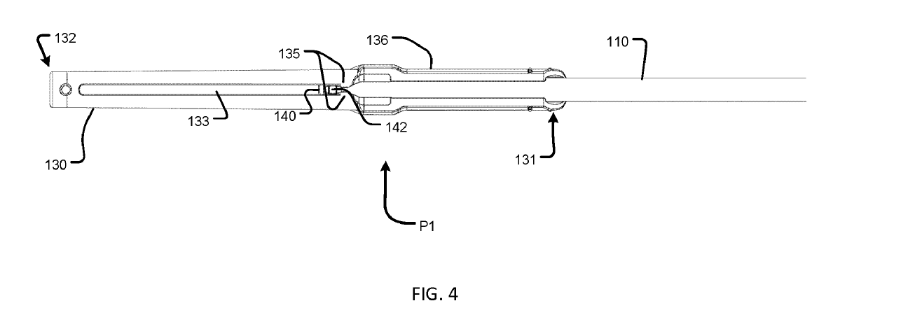

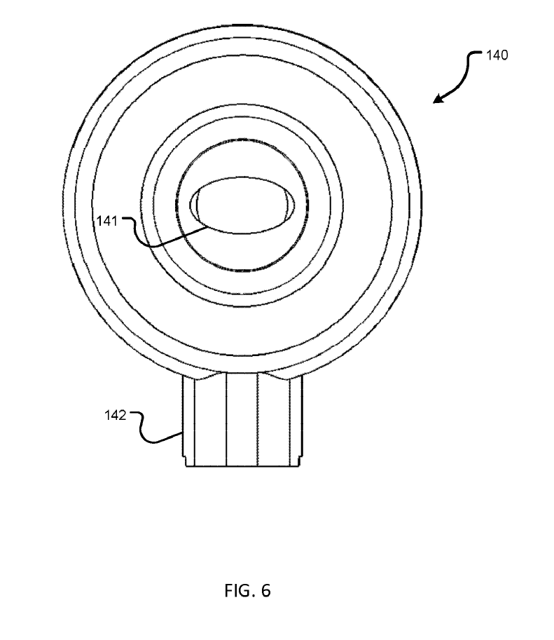

Referring to FIGS. 4-7, the stabilizer 140 of the catheter insertion device 100 may comprise a rudder 142, the outer body 130 may comprise an elongated slot 133, and the rudder 142 may be configured to slide within the slot 133 to prevent or minimize the rotation of the outer body 130 relative to the inner body 110 when the outer body 130 moves between the first position P1 and the second position P2. The outer body 130 of the catheter insertion device 100 may further comprise a stop member 135 at a distal end of the slot 133, located at a distal end of the outer body 130. The stop member 135 may be configured to abut against the rudder 142 of the stabilizer 140 to prevent the outer body 130 from moving in a proximal direction relative to the inner body 110.

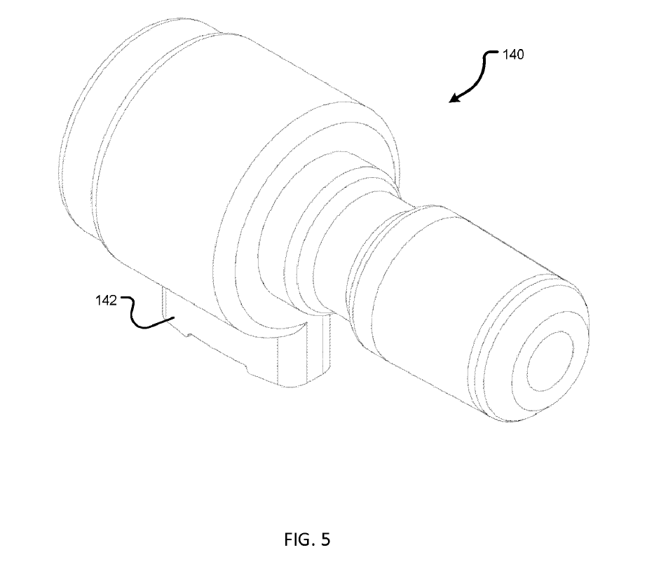

As shown in FIG. 5, the stabilizer 140 may further include a base, with the rudder 142 extending from the base, and a distal portion of the stabilizer 140 including a nose. An annular protrusion may also be disposed between the base and the nose. The nose and the annular protrusion may be configured to be received within the proximal end of the flashback chamber 114 as shown in FIG. 13. The annular protrusion may be configured to provide an interference fit with an inner surface of the flashback chamber 114 of inner body 110 for securing the stabilizer 140.

Additionally, the stop member 135 may include a pair of protrusions extending into the elongated slot 133 and form a passage to an open distal end of the slot 133. The rudder 142 of the stabilizer 140 may be configured to pass through the passage of the slot 133 when a threshold force (such as between 3N and 10N) is applied against the pair of protrusions. In this way, these protrusions may be configured to allow the outer body 130 to detach from and/or attach to the inner body 110. Thus, this configuration will provide for a stop when the user moves the outer body 130 to the second position P2. As such, when a user pulls the outer body 130 further out from this position, each protrusion 135 of the stop member 135 will elastically deform orthogonally to the slot 133 to allow enough of a gap for the outer body 130 to release from the rudder 142 of the stabilizer 140.

This aspect of the invention allows for ease of manufacturing and replacement of parts. By removing the outer body 130 from the inner body 110, the stiffening member 160, guidewire 150, and outer body 130 parts may be easily replaced if broken or damaged. Further, the rudder 142 configured to slide along the slot 133 allows the catheter insertion device 100 to remain stable throughout the movement from the first position P1 to the second position P2. That is, the rudder 142 prevents the outer body 130 from rotating around the inner body 110 while moving between the first position P1 and the second position P2.

In another aspect of the invention, the catheter insertion device 100 may include a disk 170 of elastomeric material within the stabilizer 140 configured to provide drag on the stiffening member 160 when the outer body 130 moves between the first position P1 and the second position P2. In this configuration, the aperture 141 of the stabilizer 140 is formed by the aperture of the disk 170. Preferably, the elastomeric material is silicone, but any elastomeric material known to those skilled in the art would be suitable.

However, alternative embodiments of the stabilizer 140 may also be implemented. For example, in one alternative embodiment instead of including an elastomeric disk 170 provided to produce drag on the stiffening member 160, the stabilizer aperture 141 may be shaped to produce the drag. As such, the aperture may be positioned at an angle along the longitudinal axis of the catheterization device 100, rather than straight as seen in the Figures. In another alternative embodiment, the stabilizer 140 does not include a rudder 142 and the outer body 130 does not include a slot 133, but rather the stabilizer 140 is of a shape complimentary to the internal shape of the outer body 130, such as a square shape, to provide stability during movement from the first position P1 to the second position P2.

Additionally, the outer body 130 of the catheter insertion device 100 may also comprise a guidewire actuator 137 on the distal end of the outer body configured to move the guidewire between the undeployed state and the deployed state. A distal end of the guidewire actuator 137 may include a flag configured to allow a user to move the outer body 130 between the first position P1 and the second position P2. Further, a proximal end of the guidewire actuator 137 may be an over-molded portion 136 at a distal end of the outer body 131. However, the guidewire actuator 137 may also be snap fit, press fit, welded, glued, or otherwise attached to the outer body 130.

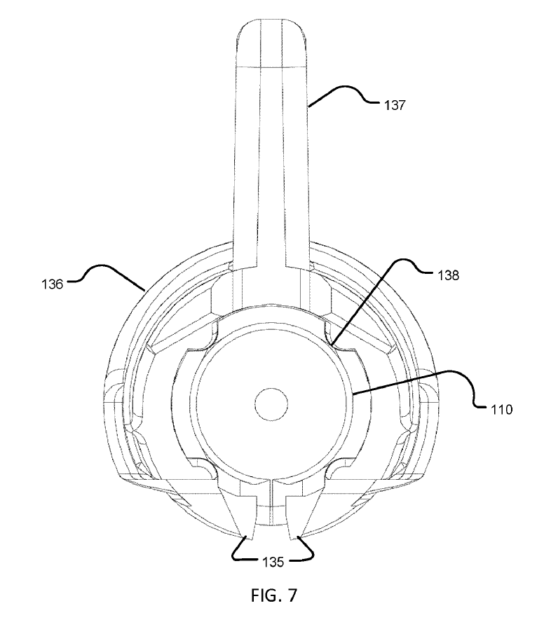

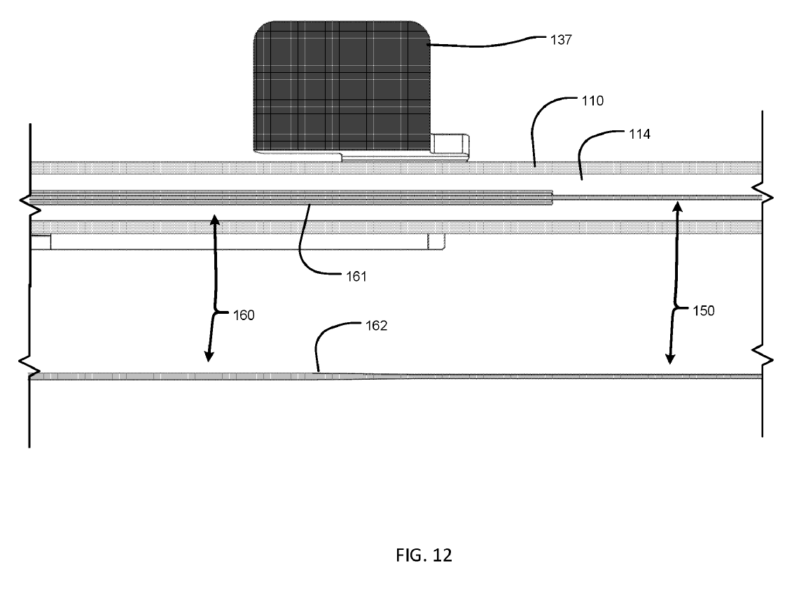

Here, a distal end of the stiffening member 160 may be arranged below the flag 137, such as seen in FIG. 12. Additionally, the guidewire actuator 137 may include at least one projection 138 extending inwardly from the outer body 130 towards the inner body 110. Preferably the guidewire actuator 137 may include a plurality of projections 138, such as the four projections 138 seen in FIG. 7. The projection(s) 138 may be configured to contact the inner body 110. This arrangement minimizes the contact points of the outer body on the inner body to create a smooth operation of movement when the outer body moves between the first position P1 and the second position P2.

Further, the arrangement between the slot 133 of the outer body, the projections 138 of the outer body, the inner body 110, and the rudder 142 of the stabilizer provides drag on the stiffening member 160 as the outer body 130 moves between the first position P1 and the second position P2. This configuration also helps to create a tactile feel to the user. As such, locating the stiffening member 160 below the flag 137 creates a stable push point allowing the user to easily move the outer body from the first position P1 and the second position P2 without buckling the guidewire 150. The flag 137 further provides for an advancement grip and may be located at the distal end 131 of the outer body or as close to the front of the device as possible to provide for shorter reach and enable the user to use the device with one hand.

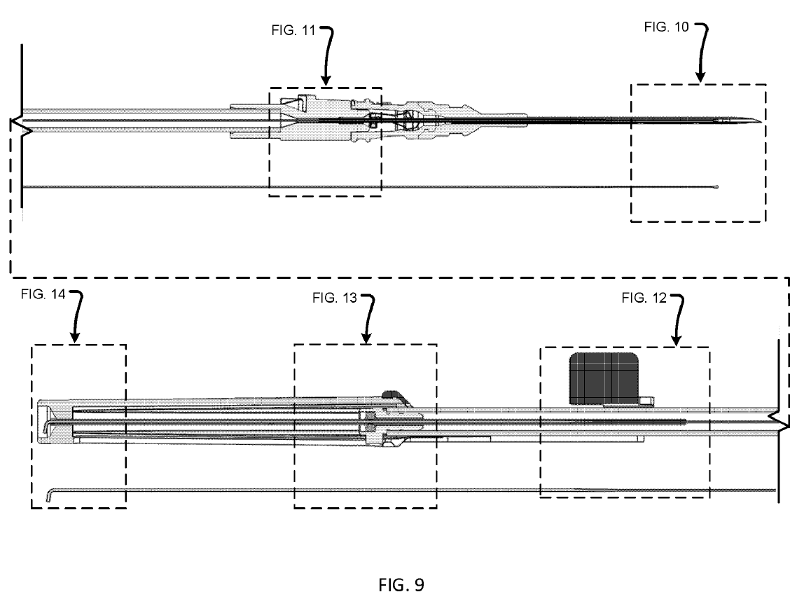

Referring to FIGS. 9-14, in embodiments of the invention, the stiffening member 160 may be a hypotube 161 surrounding the guidewire 150 or the stiffening member 160 may be a thickened portion 162 of the guidewire 150. The hypotube 161 and the guidewire 150 may be arranged to be concentric with one another and both may be fixed at the proximal end of the outer body 132. Use of the hypotube 161 as the stiffening member 160 improves the manufacturability of the device because it allows the catheter insertion device 100 to utilize the benefits of commonly made medical components or materials. Advantageously, the hypotube 161 may be made of medical grade stainless steel or other material known to those skilled in the art. Further, the hypotube 161 may be a 21-gauge tube or other size capable of forming the desired clearance described herein. The guidewire 150 may also be made of medical grade stainless steel or other material known to those skilled in the art.

The stiffening member 160 may be a thickened portion 162 of the guidewire 150. This embodiment reduces the overall number of parts and may improve the sealing feature as described above by eliminating any gap that may be formed between the stiffening member 160 and the guidewire 150 when the stiffening member 160 and the guidewire 150 are separate components. The guidewire 150 may be made of medical grade stainless steel or other material known to those skilled in the art.

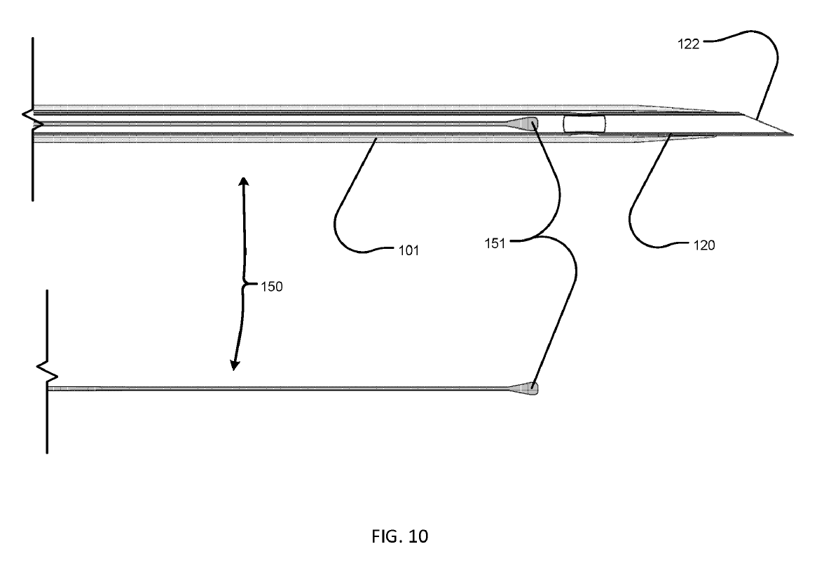

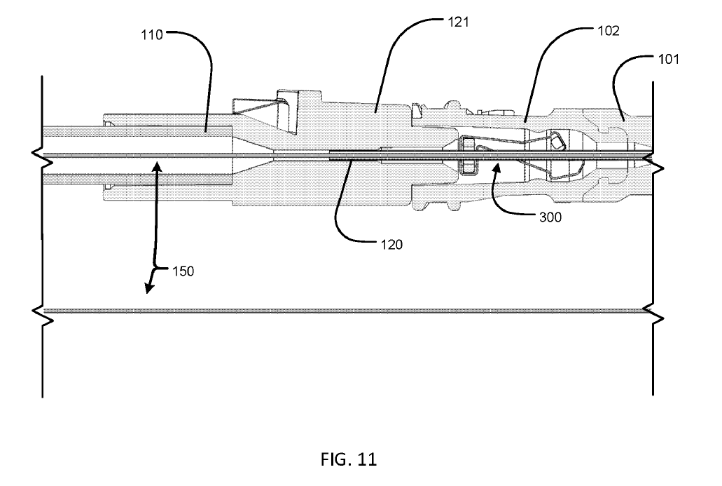

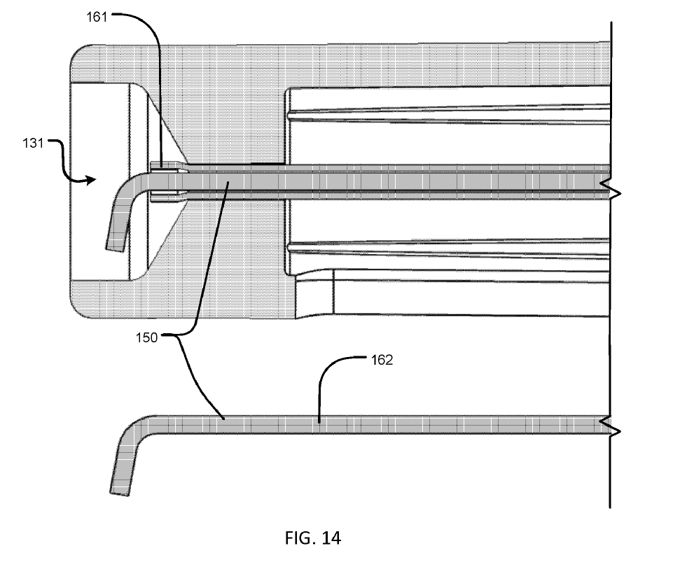

In aspects of the invention, the guidewire 150 may have a tip section (FIG. 10), a first middle section (FIG. 11), a second middle section (FIG. 12), a drag producing section (FIG. 13), and a proximal section (FIG. 14) as seen in FIG. 9. In other aspects of the invention, the diameter of the guidewire 150, except for the tip section, may be no greater than 0.0165 inches to allow for continuous pulsatile flash within the catheter insertion device 100. The guidewire 150 may extend from 0 to 3.5 centimeters from the tip 122 of the needle 120 during advancement.

When the stiffening member 160 may be a hypotube 161, the guidewire 150 has a uniform diameter from the first middle section to the proximal section. In contrast, when the stiffening member 160 may be a thickened portion 162 of the guidewire 150, the guidewire 150 has an improved stiffness profile that may increase in thickness at a portion more proximal along the length of the guidewire 150.

For example, FIG. 10 illustrates the tip section of the guidewire 150. The tip 151 may be an atraumatic tip and may be tear drop shaped, ball shaped, rounded shape, or other shape known to those skilled in the art with a 0.0165-inch diameter. The tip section may be welded into shape on the guidewire 150 or otherwise rounded through mechanical machining methods known to those skilled in the art. In either case, when the stiffening member 160 is a hypotube 161 or a thickened portion 162 of the guidewire 150, the tip section of the guidewire 150 has the same profile. After the tip section 151 of the guidewire, the guidewire 150 decreases in diameter to a first middle section of 0.0045 inches. Here, the first middle section may be configured to be floppy to ensure the tip section does not get caught on the needle tip if the guidewire 150 is retracted during use In either case, when the stiffening member 160 is a hypotube 161 or a thickened portion 162 of the guidewire 150, the first middle section of the guidewire 150 has the same profile.

FIG. 11 illustrates a portion of the guidewire 150, which transitions from the first middle section to a second middle section. Here, along this section of the guidewire 150, the first middle section tapers to the second middle section from 0.0045 inches diameter to 0.0100 inches in diameter, for example. As with the tip section, when the stiffening member 160 is either a hypotube 161 or a thickened portion 162 of the guidewire 150, the first middle section of the guidewire 150 has the same profile.

FIG. 12 illustrates a portion of the guidewire 150 as it transitions from the second middle section of the guidewire 150 to the thickened portion 162 of the guidewire 150. In this case, the guidewire 150 tapers along the second middle section from its beginning .0100-inch diameter to the thickened portion 162 of the guidewire of a thickness of .0320-inch diameter.

In contrast, when the stiffening member 160 may be a hypotube 161, the second middle section is a uniform thickness along the entire length of the second middle section and may be 0.0100 inches in diameter, for example.. However, the combined thickness of the guidewire 150 and the hypotube 161 may be 0.0320 inches in diameter, for example.

FIG. 13 illustrates a portion of the guidewire 150. In this section, when the stiffening member 160 may be a hypotube 161, the guidewire 150 may be of a thickness, such as 0.0165 inches in diameter, which is a nominal, unground diameter of the guidewire. Further, the combined thickness of the guidewire 150 and the hypotube 161 may be 0.0320 inches in diameter. In this way, the hypotube 161 may maintain a partial seal between the outer body 130, inner body 110, stabilizer 140, and hypotube 161. In contrast, when the stiffening member 160 may be a thickened portion 162 of the guidewire 150, this section in FIG. 13 may be uniformly 0.0320 inches in diameter to maintain a partial seal between the outer body 130, inner body 110, stabilizer 140, guidewire 150, and stiffening member 160.

The stabilizer 140 preferably has an elliptical aperture in shape to interfere with the stiffening member 160. For example, the elliptical aperture may have a minor diameter of 0.0260 inches and a maximum diameter of 0.0480 inches. As a result, this clearance provides a drag on the stiffening member 160 by virtue of the minor diameter being smaller than the outer diameter of the stiffening member 160 and also provides a partial seal by virtue of the maximum diameter being greater than the outer diameter of the stiffening member 160. If the stabilizer 140 had a circular aperture of the same diameter as the outer diameter of the stiffening member of 0.0320 inches, however, a full seal would be created which would not allow the catheter insertion device 100 to fill with the flash of blood.

Thus, this elliptical aperture 141 provides a clearance that is sufficient to routinely prevent blood flow from the inner body 110 chamber of the catheter insertion device 100, while still allowing air to pass through and allow hypotube 161 as the stiffening member 160 to easily slide from the first position P1 to the second position P2 Moreover, this clearance is sufficient to routinely prevent blood flow from the inner body 110 chamber of the catheter insertion device 100, while still allowing air to pass through and allow the guidewire 150 and stiffening member 160 to easily slide from the first position P1 to the second position P2 when the thickened portion 162 of the guidewire 150 is the stiffening member 160.

FIG. 14 illustrates two different views of a proximal section of the guidewire 150. In this section, when the stiffening member 160 may be a hypotube 161, the partial sealing section of the guidewire 150 may be of the same uniform thickness as the first middle section, the second middle section, and the partial sealing section, such as 0.0045 inches in diameter. Further, the combined thickness of the guidewire 150 and the hypotube 161 may be 0.0165 inches in diameter. Additionally, the hypotube 161 may be crimped or otherwise fixed to the guidewire 150. Moreover, the guidewire 150 and the hypotube 161 may be fixed to the outer body 130 at an anchor hole provided on the proximal end of the outer body 132. Adhesive such as glue or other bonding material may also be used to further fixedly secure the proximal end of the guidewire 150 and the hypotube 161 to the outer body 130. The location of the anchor hole may be axially aligned with the location of a proximal opening aperture 141 of the stabilizer.

In contrast, when the stiffening member 160 may be a thickened portion 162 of the guidewire 150, the proximal section of the guidewire 150 may be the same uniform thickness as the partial sealing section of the guidewire 150, such as 0.0165 inches in diameter. Moreover, the guidewire 150 at the thickened portion 162 may be fixed to the outer body 130 at an anchor hole provided on the proximal end of the outer body 132. Adhesive such as glue or other bonding material may also be used to further fixedly secure the proximal end of the guidewire 150 to the outer body 130. The location of the anchor hole may be axially aligned with the location of a proximal opening aperture 141 of the stabilizer.

In both embodiments, where the stiffening member 160 may be a hypotube 161 or a thickened section of the guidewire 150, the guidewire 150 may have a curved end in order to facilitate attachment to the proximal end of the outer body 132. This configuration allows for the outer body 130, the guidewire 150, and the stiffening member 160 to be able to move together as one unit with respect to the inner body 110. As described herein, the relative thickness and diameter of the stiffening member 160, the guidewire 150, and/or the stiffening member 160 may include a tolerance of 0.0003 inches or any other suitable tolerance known to a person skilled in the art.

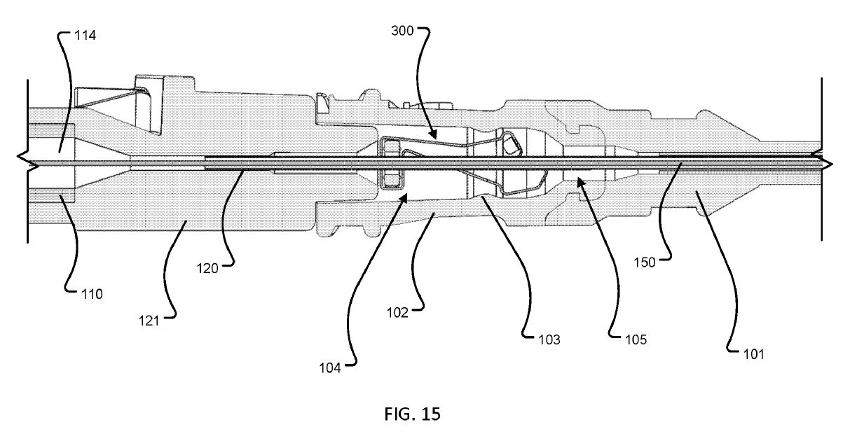

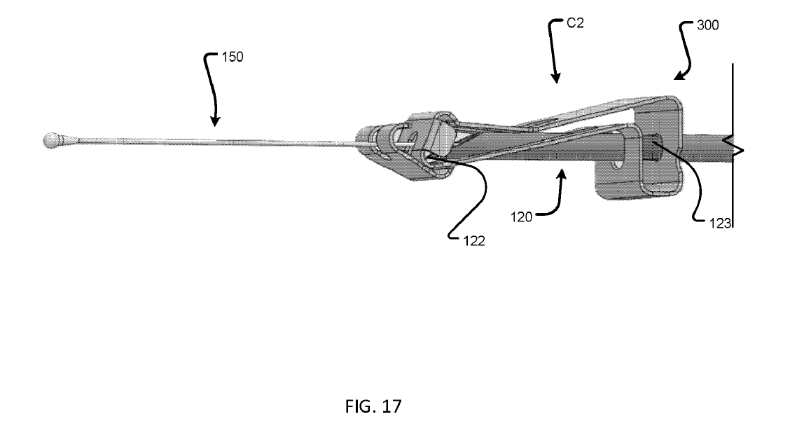

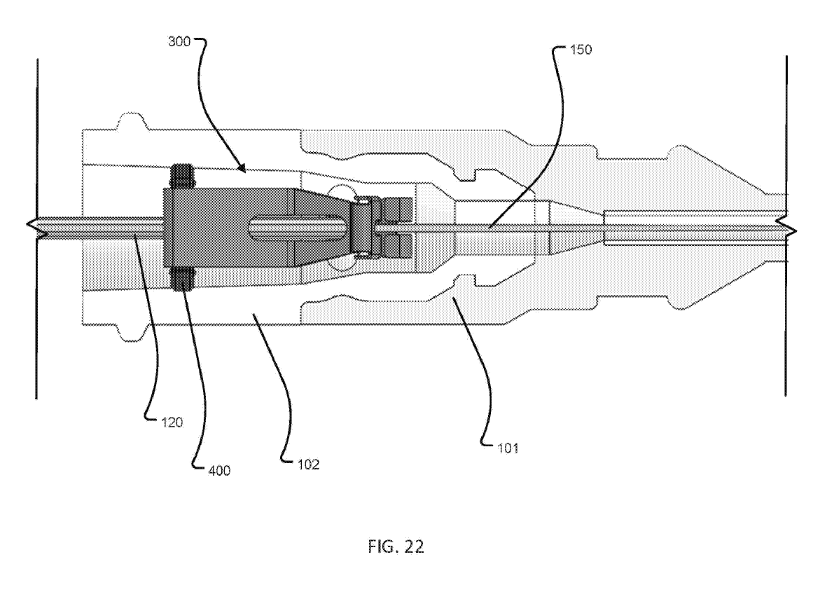

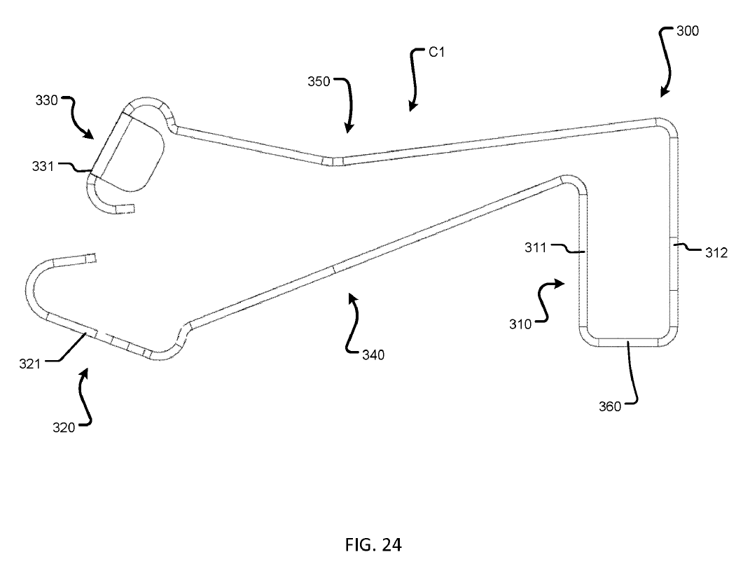

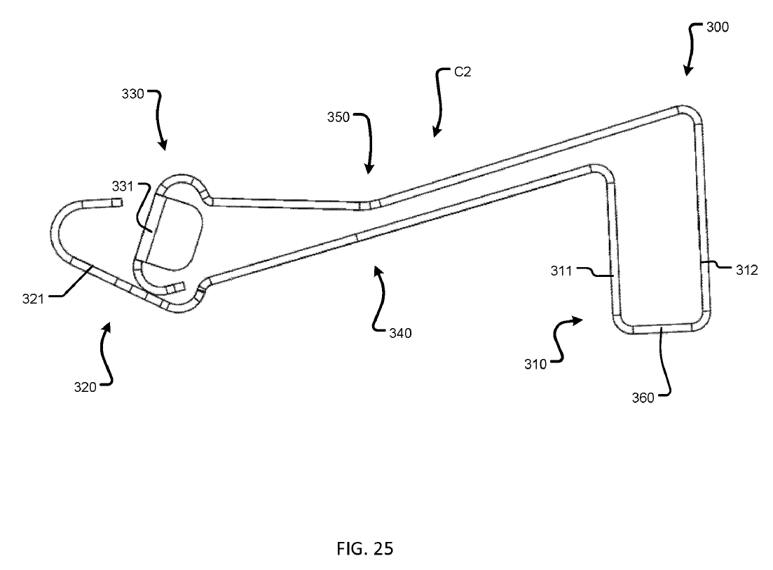

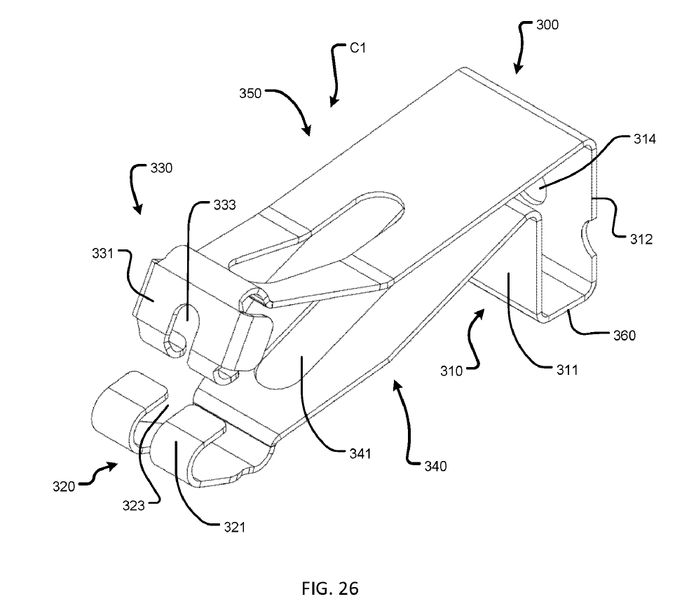

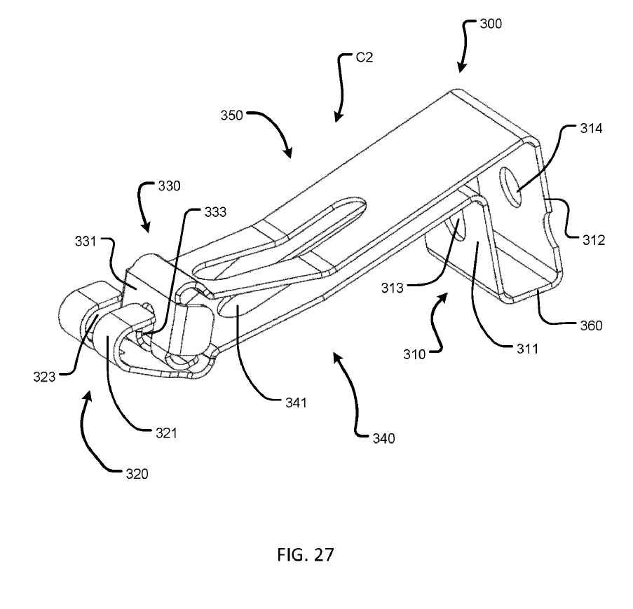

Referring to FIGS. 15-27, another aspect of the present invention relates to a safety clip 300 of the catheter insertion device 100. The safety clip 300 may include a proximal portion with a first proximal wall 311 directly connected to a second proximal wall 312 at a proximal clip bend 360. This proximal clip bend 360 may have a single curve, may be rectilinear, or may otherwise be any shape known to those skilled in the art capable of providing an elastic force. The first proximal wall 311 may have a first proximal aperture 313 and the second proximal wall 312 may have a second proximal aperture 314. Further, the second proximal wall 312 may be located more proximal than the first proximal wall 311. In this manner, the needle 120 may slide through the first proximal aperture 313 and the second proximal aperture 314.

Additionally, the safety clip 300 may have a first distal portion 320 with a first distal wall 321, which may have a first distal slot 323 for receiving the needle 120. The first distal wall 321 may be connected to a first distal face 322 by a first distal face 324. The safety clip 300 may also have a second distal portion 330 having a second distal wall 331 connected to a second distal face 332 by a second distal face 334. Further, a first distal slot 323 may extend through a portion of the first distal wall 321 and the first distal face 322, the first distal slot 323 configured to receive a guidewire of the needle.

Moreover, the safety clip 300 may have a second distal portion 330 with a second distal wall 331, the second distal wall 331 having a second distal slot 333 for receiving the needle 120. There may also be a second distal slot 333 extending through a portion of the second distal wall 331 and the second distal face 332, which may be configured to receive the guidewire of the needle. The first distal wall 321 and the second distal wall 331 may have various shapes. For example, the walls may be angular or flat such as the first distal wall 321.

The safety clip 300 may also include a first flexible clip arm 340 which may extend from the first distal portion 320 to the first proximal wall 311 and may include a first elongated medial slot 341 for receiving the needle 120. The safety clip 300 also can include a second flexible clip arm 350 extending from the second distal portion 330 to the second proximal wall 312, where the first flexible clip arm 340 and the second flexible clip arm 350 contacts the needle 120.The second clip arm 350 may also include a second elongated medial slot 351 configured to laterally receive a second portion of the needle. Moreover, the second flexible clip arm 350 may include a distal planar section 352, a proximal planar section 353 and a bend 354 such that when the safety clip 300 is in the closed configuration the second elongated medial slot 351 configured to prevent the first flexible clip arm 340 and the second flexible clip arm 350 from contacting in the closed configuration. Further, in this arrangement, the first distal portion 320 is more distal than the second distal portion 330 so that first distal wall 321 overlaps with the second distal wall 331 when the safety clip 300 is in its closed configuration. However, it may be envisioned that the first distal portion 320 may be more proximal than the second distal portion 330 so that first distal wall 321 overlaps with the second distal wall 331 when the safety clip 300 is in its closed configuration.

This design allows for the safety clip 300 to elastically move between two positions. A first, open configuration C1, and a second, closed configuration C2. It is important to note that, at rest, the safety clip 300 is configured so the first clip arm 340 and the second clip arm 350 are biased toward the closed configuration C2. Thus, the first clip arm 340 and/or the second clip arm 350 may be configured to press against the needle when in the closed configuration C2 and provide sharps protection from the needle 120. In this way, the first clip arm 340, the second clip arm 350, or both may be configured to press against the needle when in the closed configuration C2. Additionally, the first proximal aperture 313 may be coaxial with the second proximal aperture 314 and the first distal face 322 and the second distal face 332 may be configured to press against the needle when the first clip arm 340 and the second clip arm 350 are in the open configuration C1. Furthermore, the second distal face 334 may be configured to abut the first distal wall 321 when the first clip arm 340 and the second clip arm 350 in the closed configuration. Moreover, a width of the first distal slot 323 may be greater than a width of the first medial slot, and a width of the second distal slot 333 may be greater than a width of the second medial slot.

As a result, the first flexible clip arm 340 and the second flexible clip arm 350 may be configured to flex between the first, open configuration C1 and the second, closed configuration C2 to move the safety clip 300 relative to the needle. Further, the first flexible clip arm 340 and the second flexible clip arm 350 are configured to move between the open configuration C1 and the closed configuration C2 as the first flexible clip arm 340 and the second flexible clip arm 350 are biased toward the closed configuration C2 in the rest state of the safety clip 300. Additionally, the first flexible clip arm 340 and the second flexible clip arm 350 are able to move to the closed configuration C2 with or without the guidewire being advanced beyond the tip of the needle.

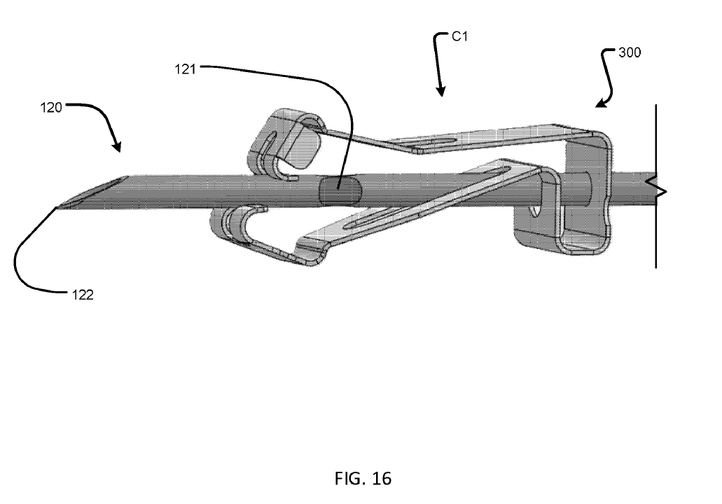

This flexing movement occurs when the first distal wall 321 and the second distal wall 331 of the safety clip 300 reach the needle tip 122 and the first proximal aperture 313 reaches a swaged portion 123 of the needle 120. Best seen in FIG. 16 this swaged portion 123 is configured to be in an oval shape and prevents the safety clip 300 from being pulled easily off the needle tip 122 as the second proximal aperture 314 of the safety clip 300 has a smaller diameter than the major outer diameter of the swaged portion 123.

For example, when the first flexible clip arm 340 and the second flexible clip arm 350 are in the open configuration C1, the safety clip 300 may be able to slide along a length of the needle 120 and the first distal wall 321 and the second distal wall 331 are spaced apart. Additionally, when the first flexible clip arm 340 and the second flexible clip arm 350 are in the closed configuration C2, the second proximal aperture 314 may be configured to engage a swaged portion 123 of the needle. Further, in the closed configuration C2, the first distal wall 321 and the second distal wall 331 overlap to cover the tip 122 of the needle 120 as the first flexible clip arm 340 and the second flexible clip arm 350 move to the rest state of the safety clip 300 in the second configuration C2. Moreover, the first distal slot 323 and the second distal slot 333 may be configured to allow the first flexible clip arm 340 and the second flexible clip arm 350 to move to the closed configuration C2 with a guidewire advanced beyond the tip of the needle and may allow a guidewire to be retracted while in the closed configuration C2.

Thus, due to the first flexible clip arm 340 and the second flexible clip arm 350 being biased toward the closed configuration C2, the first distal wall 321 and the second distal wall 331 may be configured to cover a needle tip 122 to protect the user from injury. As seen in the Figures, to form such a cover, the first distal wall 321 is more distal than the second distal wall 331. However, in other aspects of the invention, the second distal wall 331 may be more distal than the first distal wall 321. As seen in the Figures, the first flexible clip arm 340 and the second flexible clip arm 350 do not cross each other in either the open configuration C1 or the closed configuration C2.

Accordingly, this design allows the safety clip 300 to easily be made in one piece. The safety clip 300 may be made by any known manufacturing method known to those skilled in the art such as 3D printing, casting, forging, die forming, and/or the like. Additionally, the clip may be made of any flexible, resilient or elastic material, such as a metal including aluminum or stainless steel, or plastic, capable of providing enough elastic force for the safety clip 300 to move between the open configuration C1 and the closed configuration C2. For example, the safety clip 300 may be manufactured of a single piece of metal, stamped, and then bent into shape.

The safety clip 300 may be used in a medical device which utilizes a needle 120 such as a catheter insertion device 100, a guidewire advancement device, syringe device, or any other device known to those skilled in the art. For example, to prevent a catheter 101 from being removed inadvertently from the catheter insertion device 100 before the safety clip 300 covers the needle tip 122 in its closed configuration C2, the catheter hub 102 may include a protrusion or protrusion 103. This projection 103 is configured so that the internal diameter of the catheter hub 102 interferes with the safety clip 300 when the safety clip 300 is in its open configuration C1 and does not interfere with the safety clip 300 when the safety clip 300 is in its closed configuration C2. This feature also allows the catheter 101 to move simultaneously with the safety clip 300 when the user inserts the catheter 101 into the vessel 200 of the patient. As such, the projection 103 may define the hub 102 into a proximal cavity and a distal cavity.

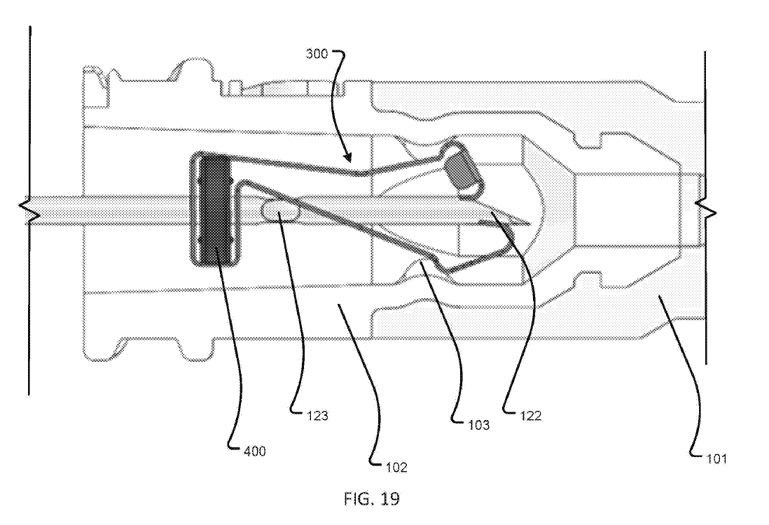

That is, in FIG. 19, the safety clip 300 is in the open configuration C1 where its first distal wall 321 and the second distal wall 331 are spread apart at a radial distance beyond the internal diameter of the catheter hub 102 at the protrusion 103. When the safety clip 300 reaches the end of the needle 120, in FIG. 28, the needle 120 flexes to the closed configuration C2 compressing the first distal wall 321 and the second distal wall 331 together in such a way to allow the catheter 101 to freely move with respect to the needle 120, the safety clip 300, and the medical device. Thus, as the catheter 101 is inserted into the vessel 200 of the patient the safety clip 300 will also cover the needle tip to protect the user from injury.

Additionally, the safety clip 300 may include a one-piece body. Moreover, the first distal portion 320 of the safety clip 300 may include a first curved elbow 325 connecting the first clip arm 340 to the first distal wall 321, and the second distal portion 330 of the safety clip 300 may include a second curved elbow 335 connecting the second clip arm 350 to the second distal wall 331. Further, the first and second curved elbows may be configured to contact a bump 103 portion within a hub of the catheter 101 to prevent removal of the safety clip 300 from inside the hub when in the open configuration. The safety clip 300 may also be configured to be removed from the hub of the catheter 101 when the first clip arm 340 and the second clip arm 350 are in the closed configuration.

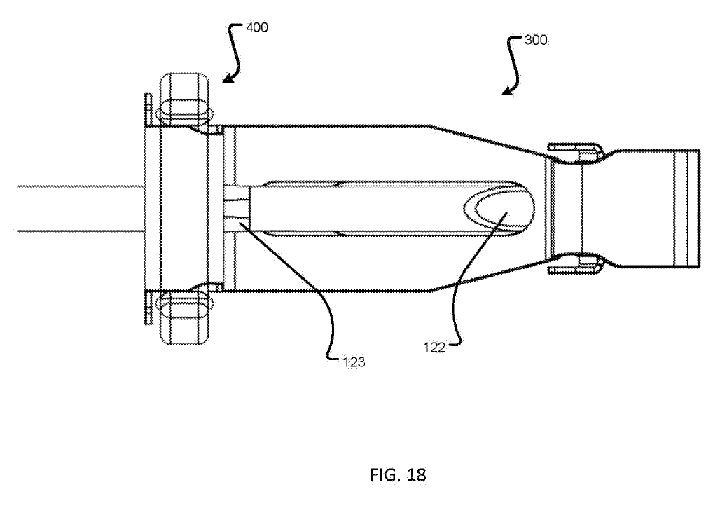

Referring to FIGS. 18-23, in another aspect of the invention, the safety clip 300 may include a flexible keeper 400 made of silicone or another elastomeric material between the first proximal wall 311 and the second proximal wall 312. The operation of the safety clip 300 and the structure of the safety clip 300 with and without the keeper 400 are substantially the same. However, the keeper 400 may be configured to ensure the safety clip 300 remains properly retained within the catheter hub 102 as the catheter 101 is inserted into the vessel 200 of the patient and removed from the catheter insertion device 100. The keeper 400 may thus be configured to provide frictional resistance against an inner surface of the catheter hub 102 to prevent the safety clip 300 from pulling out of the hub 102 absent application of a threshold force.

For this purpose, the catheter hub 102 initially has a larger inner diameter than the outer diameter of the keeper 400, but the inner diameter of the catheter hub 102 slowly decreases in diameter as the keeper 400 is pushed into the catheter hub 102 until the outer diameter of the keeper 400 is slightly larger than the inner diameter of the catheter hub 102. This arrangement creates friction or drag between the keeper 400 and the catheter hub 102 to help ensure the safety clip 300 stays with the catheter 101 until the safety clip 300 can activate towards is closed configuration C2 to cover the needle tip 122 as the catheter 101 is being inserted into a vessel of a patient and the medical device is being removed from the catheter 101.

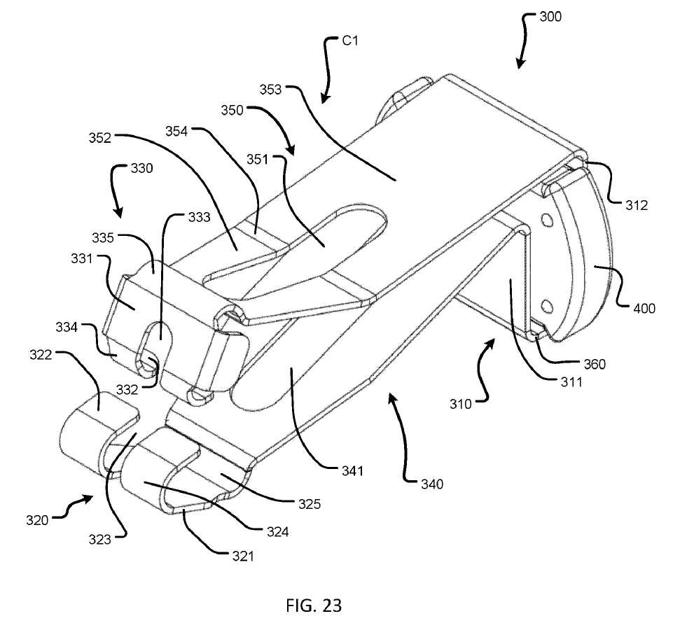

The keeper 400 may be of one piece that fits within the space between the first proximal wall 311 and the second proximal wall 312 of the safety clip 300 as seen in FIG. 23 or could include projections integral with the safety clip 300 extending from the first proximal wall 311 and/or second proximal wall 312 of the safety clip 300. It is important to note that the force to pull the swaged portion 125 of the needle 120 through the internal diameter of the second proximal aperture 314 is much greater than the force to pull the keeper 400 from the internal diameter of the catheter 101.

The keeper 400 may also include a through-hole configured to slidably receive the needle. Further, a diameter of the through-hole may be greater than a diameter of the first proximal aperture 313 and a diameter of the second proximal aperture 314. Additionally, in embodiments where the safety clip 300 uses a keeper 400, the friction or drag force of the keeper 400 should not be large enough so that it would pull on the catheter 101 during removal of the medical device after the catheter 101 has been fully inserted into the blood vessel 200 of the patient. However, a person skilled in the art would be able to determine through routine experimentation the desired interference fit and material capable of creating the desired results of the keeper 400 as described herein.

To assemble the catheter insertion device 100 with the safety clip 300 the keeper 400 is pushed into a slot of the safety clip 300 then a tool is used to spread open the first clip arm 340 and the second clip arm 350. The back end of the needle 120 is then slid into the safety clip 300. In this way, the back end of the needle first slides past the first distal slot 323, then past the second distal slot 333, then through the first elongated medial slot 341, past the second elongated medial slot 351, through the first proximal aperture 313, and finally through the second proximal aperture 314 of the safety clip 300. The safety clip 300 will slide along the length of the needle 120 and will rest in the center of the length of the needle 120.

Here, the needle 120 and safety clip 300 and keeper 400 assembly is removed from the tool used to spread open the first clip arm 340 and the second clip arm 350. At this point, the needle 120 is glued, or otherwise affixed to the needle hub. A catheter 101 may then be slid over the needle tip 122 of the catheter insertion device 100 which causes the first curved elbow 325 and the second curved elbow 335 to contact the protrusion 103 of the catheter 101. Pushing on the catheter 101, slides the safety clip 300 along the needle 120 until the safety clip 300 contacts the needle hub 121. At this point, the needle hub 121 pushes back onto the safety clip 300 and causes the arms of the safety clip 300 to elastically compress until the first distal wall 321 and the second distal wall 331 have moved past the protrusion 103 at which point the safety clip 300 uncompresses back to its original geometry. Thereafter, the safety clip 300 will move simultaneously with the catheter 101 along the length of the needle 120 until the safety clip 300 activates. This structure of the safety clip 300 thus creates an over the needle catheter 101 that conceals the safety clip 300 during catheter insertion.

FIGS. 15-22 show the safety clip 300 in operation with the catheter insertion device 100. In particular, catheter insertion may begin with the catheter insertion device 100 in the first position P1 seen in FIG. 1. Then, after preparing the patient for needle insertion, the user (e.g., practitioner or clinician) may insert the tip 122 of the needle 120 at approximately a 30° insertion angle (γ) with respect to the surface of the skin to access the vessel 200 of the patient as illustrated in FIG. 3. At this point, blood will flash back into the inner body 110 before the advancement of the guidewire 150. This blood flashback provides indication to the user that the needle 120 has accessed the blood vessel 200.

From here, the user moves the outer body 130 from the first position P1, where a guidewire tip 151 is within the needle 120, to a second position P2, where the guidewire tip 151 is advanced into the vessel 200 of the patient. This movement may be done with one hand by pushing continuously on the flag 137 at a push point. The guidewire tip 151 forms an atraumatic end and has sufficient elasticity to bend so when the guidewire tip 151 hits the back wall of the vessel 200, the guidewire 150 will bend from the 30° insertion angle and move substantially parallel into the artery to form a guide for the catheter 101.

At the same time, when the outer body 130 moves between the first position P1 and the second position P2, the outer body 130 slides over the inner body 110 and the guidewire 150 and a stiffening member 160 fixed to the proximal end of the outer body 132 simultaneously move through an inner portion 113 of the inner body 110. At this point, the guidewire 150 and the stiffening member 160 extend through an aperture 141 of a stabilizer 140 fixed to a proximal end of the inner body 112, and the stiffening member 160 is configured to stabilize the guidewire 150 when the outer body 130 moves from the first position P1 to the second position P2.

From here, the user may advance the catheter 101 forward to insert it into the vessel by pushing the catheter hub 102 forward over the needle until the catheter hub 102 is fully advanced into the insertion site. As the user moves the catheter 101 forward, a safety clip 300 within the catheter 101 will slide along the length of the needle 120 and be in the first configuration C1 as seen in FIG. 19.

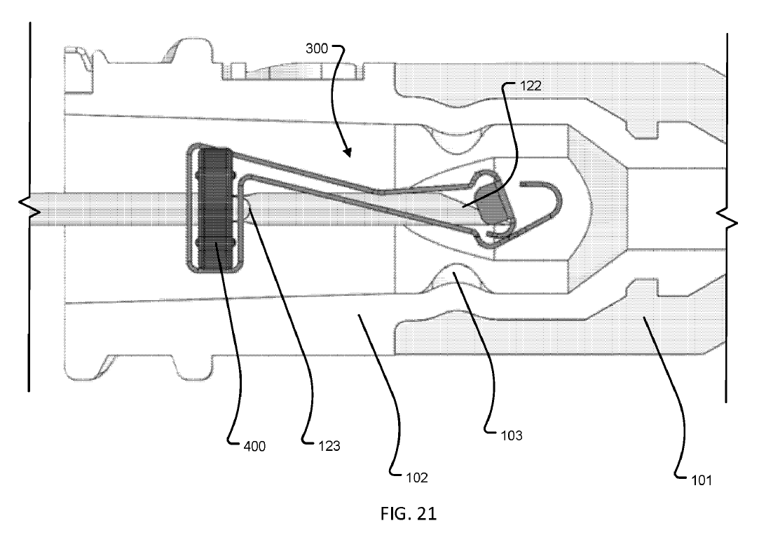

At this point, the needle 120 may be retracted to remove it from the catheter 101. As the needle 120 is retracted and pulled fully out of the catheter hub 102, the needle tip 122 will be retracted until the arms of the safety clip 300 are able to activate and the distal walls (321, 331) will begin to close around the needle tip 122 to protect the user from injury. Moreover, as the distal walls (321, 331) slide down the beveled edge, the first flexible arm 340 and the second flexible arm 350 move closer together such that the second elongated medial slot 351 laterally receives a portion of the needle 120. After the safety clip 300 is fully activated, the safety clip 300 will be in the second configuration C2 as seen in FIG. 21.

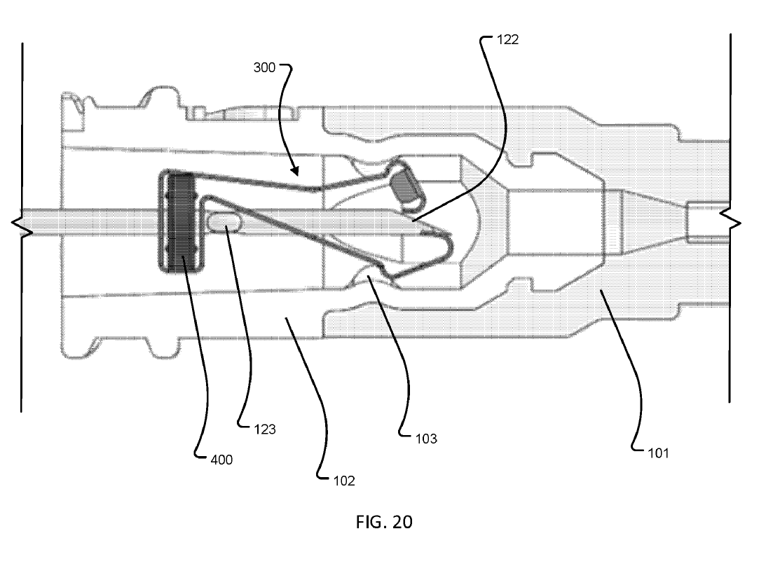

For example, when the first clip arm 340 and the second clip arm 350 are in the first configuration C1, the safety clip 300 may be able to slide along a length of the needle 120 and the first distal wall 321 and the first distal wall 331 are spaced apart. Further when the first clip arm 340 and the second clip arm 350 are in the first configuration C1, the first curved elbow 325 and the second curved elbow 335 are engaged with the projection 103 of the catheter hub 102 as the catheter 101 is inserted into the vessel 200 of the patient. When the user begins to retract the needle after catheterization, for example, after the catheter is fully advanced into the insertion site in the vessel 200, the needle will be moved proximally relative to the safety clip 300 to a point as seen in FIG. 19. At this point, the safety clip 300 is still in the open configuration C1. However, as the user continues to retract the needle, the safety clip 300 will activate at which point, the first distal wall 321 and the second distal wall 331 are located approximately at the beginning of the beveled tip 122 of the needle, as seen in FIG. 20. As the user pulls the catheter insertion device 100 back, the first distal wall 321 and the second distal wall 331 of the safety clip 300 will slide down the beveled tip 122 of the needle and activate the first clip arm 340 and the second clip arm 350 to begin to close the safety clip 300 around the needle 120 as the needle is further retracted because both the first and second flexible clip arms 340, 350 are biased to move toward each other.

Thus, after the needle 120 is fully removed from the catheter, the first clip arm 340 and the second clip arm 350 is in the second configuration C2 as seen in FIG. 21. Additionally, when the first clip arm 340 and the second clip arm 350 are in the second configuration C2, the first proximal aperture 313 may cross over a swaged portion 123 of the needle and the second proximal aperture 314 may then engage the swaged portion 123 of the needle 120 such that the needle 120 is prevented from moving further. Thus, in this second configuration C2, the first distal wall 321 and the first distal wall 331 may be configured to cover a needle tip 122 to protect the user from injury. To form such a cover, the first distal wall 321 is more distal than the first distal wall 331. However, in other aspects of the invention, the first distal wall 331 may be more distal than the first distal wall 321. The first clip arm 340 and the second clip arm 350 do not intersect in either the first configuration C1 or the second configuration C2.

The safety clip 300 also has slots (323, 333), which are larger than the guidewire 150 in order to allow the arms of the safety clip 300 to activate and still close around the needle tip 122 while the guidewire 150 may extend through the needle tip 122. From here, catheter insertion is complete, and the user may pull back on the catheter insertion device 100 and remove the needle 120 from the catheter insertion device 100 with the safety clip 300 activated.

Moreover, when the device is in the closed configuration C2, the first curved elbow 325 and the second curved elbow 335 will also clear the projection 103 of the catheter hub 102. At this point, the catheter insertion device 100 and guidewire 150 assembly are ready for removal. The user may also continue to pull back on the catheter insertion device 100. Because the safety clip 300 has activated, the needle 120 and the safety clip 300 are free to be removed from the catheter 101 with the safety clip fully in the closed configuration C2 and covering the tip of the needle. In embodiments with a safety clip 300 including a keeper 400, pulling back on the catheter insertion device 100 will cause a slight pop to be felt as the keeper 400 is released due to the interference between the keeper 400 and the catheter hub 102. The user may continue to freely pull back the catheter insertion device from the catheter 101 until the needle 120 with its tip 151 covered by the safety clip 300 is entirely removed. At this point, the medical device may be discarded into a sharps bin for disposal, while the catheter 101 is left in the vessel 200 of the patient for treatment.

Additionally, when the first clip arm 340 and the second clip arm 350 are in the first configuration C1, the safety clip 300 may exert a first force on the needle 120 tending to return the safety clip 300 back to the second configuration C2. This feature of the safety clip 300 ensures that the safety clip 300 has a resting position intended to cover the needle tip to prevent injury and intended to be disengaged from the catheter hub 102. In contrast, the first configuration C1 ensures the safety clip 300 will actively hold onto the catheter hub 102 while tending to want to move towards the second configuration C2 once the safety clip 300 activates over the needle tip 122.

In one aspect of the invention, the inner body 110 may be made from medical grade polyurethane, polyvinylchloride (PVC), and/or other materials known to those skilled in the art. The inner body 110 may include indicia to indicate where the guidewire 150 is positioned in relation to the needle tip. For example, indicia of “0” indicates the needle tip is located at the needle tip, while indicia of “1” indicates the needle tip is located 1 unit past the needle tip. In aspects of the invention, these units may be a centimeter, an inch, or another preferred measurement known to those skilled in the art. In an aspect of the invention, the inner body 110 may extend from 3.5 inches to 6 inches in length, for example. However, a longer length is preferred because a longer length reduces the rate at which the inner portion 113 of the inner body 110 may fill with blood during continuous blood flashback.

In one aspect of the invention, the outer body 130 may be over-molded from clear polycarbonate and/or other materials known to those skilled in the art. This design allows the user to see the pulsatile blood return into the inner portion 113 of the inner body 110 as the outer body 130 moves from the first position P1 to the second position P2. The flag 137 of the outer body bmay be made of high-density polyethylene (HDPE) and/or other materials known to those skilled in the art. The flag 137 may be black, blue, green, or any other color to contrast the flag 137 from the rest of the outer body 130 for easy user manipulation.

While the catheter insertion device has been described in terms of what may be considered to be specific aspects, the present disclosure is not limited to the disclosed aspects. Additional modifications and improvements to the catheter insertion apparatus may be apparent to those skilled in the art. Moreover, the many features and advantages of the disclosure are apparent from the detailed specification, and thus, it is intended by the appended claims to cover all such features and advantages of the disclosure which fall within the spirit and scope of the disclosure.

Further, it is not desired to limit the disclosure to the exact construction and operation illustrated and described, and accordingly, all suitable modifications and equivalents may be resorted to, falling within the scope of the disclosure. Accordingly, the present disclosure should be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed is:

1. A catheter insertion device comprising:
an inner body defining a continuous pulsatile blood flashback chamber;
a needle hub having a proximal end attached to a distal end of the inner body;
a hollow introducer needle extending from a distal end of the needle hub, the introducer needle having a tip configured to pierce a wall of a blood vessel;
a catheter configured to slidably fit over the introducer needle, the catheter extending from a catheter hub;
an outer body configured to slidably receive a portion of the inner body;
a guidewire coupled to a proximal end of the outer body; and
a stiffening member extending from the proximal end of the outer body;
wherein the outer body is further configured to move between a first position in which the guidewire is at an undeployed state and a second position in which the guidewire is at a deployed state;
wherein a tip of the guidewire is located within the needle when the outer body is at the first position; and
wherein the tip of the guidewire is advanced beyond the tip of the needle when the outer body is at the second position.

2. The catheter insertion device of claim 1, wherein the guidewire and the stiffening member simultaneously move when the outer body moves between the first position and the second position, such that the stiffening member stabilizes a portion of the guidewire.

3. The catheter insertion device of claim 1, further comprising a stabilizer attached to a proximal end of the inner body, the stabilizer configured to receive the stiffening member and provide friction to the stiffening member when the outer body moves between the first position and the second position.

4. The catheter insertion device of claim 3, wherein the stabilizer comprises an elastomeric disk having an elliptical or oval shaped aperture configured to receive the stiffening member and provide friction to the stiffening member when the outer body moves between the first position and the second position.

5. The catheter insertion device of claim 3, wherein the stabilizer comprises a rudder, the outer body comprises an elongated slot, and the rudder is configured to slide within the slot to prevent or minimize rotation of the outer body relative to the inner body when the outer body moves between the first position and the second position.

6. The catheter insertion device of claim 5, wherein the outer body further comprises a stop member at a distal end of the elongated slot configured to abut the rudder of the stabilizer to prevent the outer body from moving proximally relative to the inner body.

7. The catheter insertion device of claim 6, wherein the stop member includes a pair of protrusions extending into the slot, the pair of protrusions forming a passage to an open distal end of the slot; and wherein the rudder of the stabilizer is configured to pass through the passage of the slot upon application of a threshold force against the pair of protrusions to allow the outer body to detach from and/or attach to the inner body.

8. The catheter insertion device of claim 3, wherein the stiffening member comprises a hypotube configured to receive the portion of the guidewire.

9. The catheter insertion device of claim 3, wherein the stiffening member comprises a proximal portion of the guidewire having a diameter that is larger than a diameter of a distal portion of the guidewire.

10. The catheter insertion device of claim 1, wherein a distal end of the outer body comprises a guidewire actuator configured to move the guidewire between the undeployed state and the deployed state; wherein a distal end of the stiffening member is positioned below the guidewire actuator to create a push point for the user.

11. The catheter insertion device of claim 10, wherein the guidewire actuator comprises at least one projection configured to contact the inner body to minimize friction when the outer body is moved between the first position and the second position.

12. The catheter insertion device of claim 1, further comprising a safety clip configured to provide sharps protection to the tip of the introducer needle upon removal of the catheter from the introducer needle, wherein the safety clip further comprises a keeper configured to provide frictional resistance against an inner surface of the catheter hub to retain the safety clip inside the catheter hub.

13. The catheter insertion device of claim 12, wherein the catheter hub further comprises a bump configured to prevent the safety clip from being removed from the catheter hub in a first configuration and configured to allow the safety clip to be removed from the catheter hub in a second configuration.

14. A method of operating a device capable of continuous pulsatile blood flash to advance a guidewire fixed to a distal end of an outer body of the device into a vessel of a patient, the method comprising:
moving the outer body of the device to a first position;
inserting a needle attached to a proximal end of an inner body of the device into the vessel of the patient;
moving the outer body from the first position, where a tip of the guidewire is within the needle, to a second position, where the tip of the guidewire is advanced into the vessel of the patient;
wherein when the outer body moves between the first position and the second position, the outer body slides over the inner body and the guidewire and a stiffening member fixed to the distal end of the outer body simultaneously moves through an inner portion of the inner body;
wherein the guidewire and the stiffening member extend through a hole of a stabilizer fixed to a distal end of the inner body; and
wherein the stiffening member is configured to stabilize the guidewire when the outer body moves from the first position to the second position.

15. The method of claim 14, wherein the stiffening member comprises a hypotube configured to receive the portion of the guidewire.

16. The method of claim 14, wherein the stiffening member comprises a portion of the guidewire having a diameter that is larger than a diameter of a distal portion of the guidewire.

17. The method of claim 14, wherein the device further comprises an elastomeric disk having an elliptical or oval shaped aperture configured to receive the stiffening member and provide friction to the stiffening member when the outer body moves between the first position and the second position.

18. The method of claim 14, wherein the stabilizer comprises a rudder, the outer body comprises an elongated slot, and the rudder is configured to slide within the slot to prevent or minimize rotation of the outer body relative to the inner body when the outer body moves between the first position and the second position.

19. The method of claim 14, wherein the device further comprises a safety clip configured to provide sharps protection to the tip of the introducer needle upon removal of the catheter from the introducer needle, wherein the safety clip further comprises a keeper configured to provide frictional resistance against an inner surface of the catheter hub to retain the safety clip inside the catheter hub.

20. The method of claim 19, wherein when the safety clip reaches a bevel of a tip of the needle, the safety clip activates and allows the first clip arm and the second clip arm to move from the first configuration to the second configuration.
